(19) United States
(12) Patent Application Publication
(10) Pub. No.: US 2010/0324480 A1
(43) Pub. Date: Dec. 23, 2010

(54) AUTOMATIC INJECTION SYRINGE ASSEMBLY

(76) Inventor: Thomas Chun, Northridge, CA (US)

Correspondence Address: ITALIA IP, 3500 WEST OLIVE AVE., SUITE 300, BURBANK, CA, 91505, US

(21) Appl. No.: 12/486,356

(22) Filed: Jun. 17, 2009

(51) Int. Cl. A61M 5/31; A61M 5/20

(52) U.S. Cl. 604/89; 604/135

(57) ABSTRACT

An apparatus for applying medicament delivery pressure to a plunger pad of a plunger of a syringe includes a spring having a first end and a second end, wherein the spring first end is operable to abut the plunger pad. The assembly also includes a shell including an interior and a first step therein. A cap is disposed within the shell interior. The cap includes a surface for engaging the spring second end. The cap is movable from a first position, wherein the shell first step retains the cap in releasable engagement with the plunger such that the spring is compressed between the cap surface and plunger pad, and a second position, wherein the cap disengages the shell first step such that the cap disengages the plunger such that the spring expands to move the barrel and needle forward as well as deliver medicament delivery pressure to the plunger pad.

### BACKGROUND OF INVENTION

This application relates generally to an automatic injection syringe assembly. More specifically, this application relates to an automatic injection syringe assembly that allows a medicament to be introduced into the body through the skin.

### SUMMARY

Providing injections of medicaments to a patient is a highly successful mode of treatment. However, using a typical syringe often requires great skill and care so as to avoid injecting too deep, scratching the patient with the needle, and the like. Moreover, having the patient inject himself or herself provides the same challenges in addition to the general nervousness or anxiety frequently involved with injecting oneself. The present application discloses a automatic injection syringe assembly that decreases the skill and steady-hand that injections typically require.

In one embodiment, a syringe assembly comprises: a barrel including a forward end and a rear end and defining a reservoir for containing a medicament; a hollow needle coupled to the barrel forward end and in fluid communication with the reservoir; a plunger including a first end positioned within the reservoir and a pad for receiving medicament delivery pressure for causing the plunger to move within the reservoir to expel medicament from the reservoir; a spring including a first end and a second end, wherein the spring first end abuts the plunger pad; a cap including an interior surface for engaging the spring second end, the cap movable from a first position, wherein the cap is in releasable engagement with the plunger and wherein the spring is compressed between the cap interior surface and the plunger pad, and a second position, wherein the cap disengages the plunger such that the spring is capable of expanding and thereby provide a medicament delivery pressure to the plunger pad; a needle shield positioned near the barrel forward end, the needle shield movable from a retracted position to a needle protecting position; and a second spring including a first end engaging the barrel and a second end engaging the needle shield, the second spring capable of biasing the needle shield to the needle protecting position.

In another embodiment, an apparatus for applying a medicament delivery pressure to a plunger pad of a plunger of a syringe, the apparatus comprises: a spring including a first end and a second end, wherein the spring first end is operable to abut the plunger pad; a shell including an interior and a first step therein; and a cap disposed within the shell interior, the cap including a surface for engaging the spring second end, the cap movable from a first position, wherein the shell first step retains the cap in releasable engagement with the plunger such that the spring is compressed between the cap surface and plunger pad, and a second position, wherein the cap disengages the shell first step such that the cap disengages the plunger such that the spring expands to move the barrel and needle forward as well as deliver medicament delivery pressure to the plunger pad.

In yet another embodiment, a syringe assembly comprises: a barrel including a forward end and a rear end and defining a reservoir within which a medicament may be contained; a hollow needle coupled to the barrel forward end and in fluid communication with the reservoir; a plunger including a first end positioned within the reservoir and a pad for receiving medicament delivery pressure for causing the plunger to move within the reservoir to expel medicament from the reservoir; a spring including a first end and a second end, wherein the spring first end abuts the plunger pad; a rear shell portion including an interior and a first step therein; a cap disposed within the shell interior, the cap including a surface for engaging the spring second end, the cap movable from a first position, wherein the shell first step retains the cap in releasable engagement with the plunger such that the spring is compressed between the cap surface and plunger pad, and a second position, wherein the cap disengages the shell first step such that the cap disengages the plunger such that the spring expands to move the barrel and needle forward as well as deliver medicament delivery pressure to the plunger pad; a front shell portion, the front shell portion including an interior volume, a second step, and a aperture, wherein at least a portion of the needle extends through the front shell aperture when the barrel and needle are moved forward by the spring; a second spring including a first end and a second end, wherein the second spring first end abuts the barrel; a needle shield disposed within the aperture of the front shell, the needle shield including an aperture for allowing the needle to pass therethrough, and a shoulder for engaging the second spring second end, the needle shield movable from a retracted position, wherein the needle shield is encased by the front shell portion, and a second position, wherein at least a portion of the needle shield extends through the front shell aperture and encompasses at least a portion of the needle extending through the front shell aperture, wherein the second spring biases the needle shield in the second position after the barrel and needle are moved forward by the spring.

### BRIEF DESCRIPTION OF THE DRAWINGS

The drawings, when considered in connection with the following description, are presented for the purpose of facilitating an understanding of the subject matter sought to be protected.

FIG. 1 is a cross-sectional view of the syringe assembly of FIG. 1 showing the syringe assembly prior to an injection;

FIG. 2 is a cross-sectional view of the syringe assembly of FIG. 1 showing the syringe assembly after an injection and after the assembly has been withdrawn from a patient;

FIG. 3 is an exploded schematic view of the syringe assembly of FIG. 1; and

FIG. 4 is an alternative syringe assembly showing the assembly with a LyoTip™ barrel arrangement.

### DETAILED DESCRIPTION

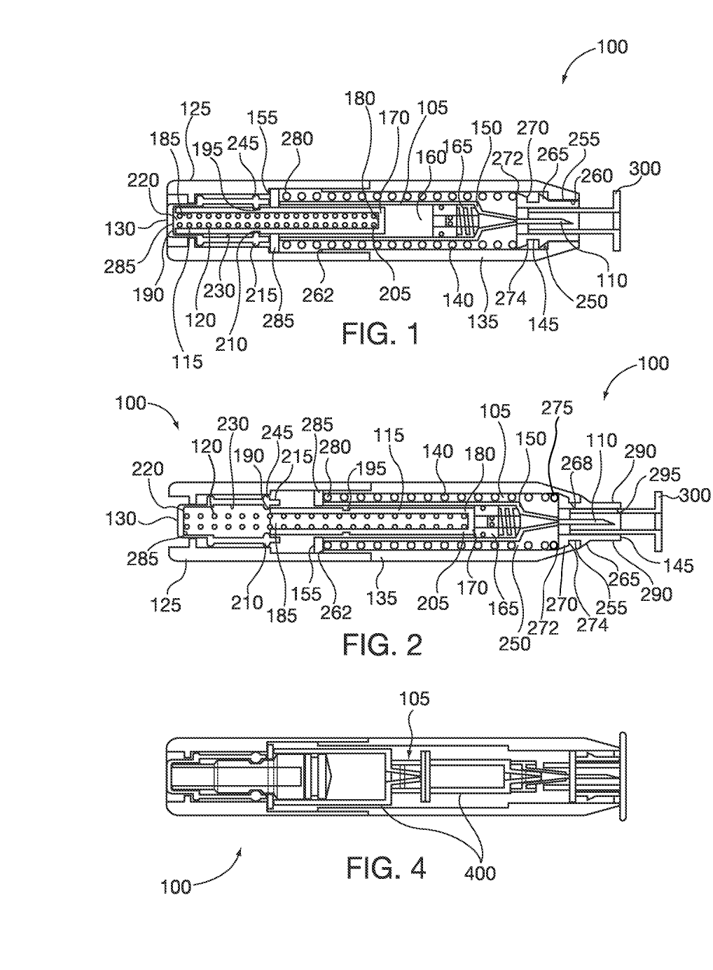

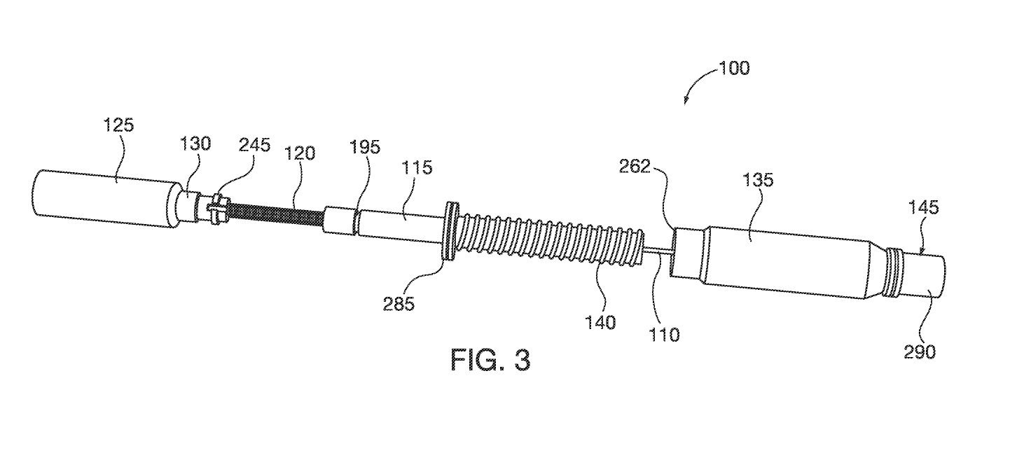

Referring now to FIGS. 1-3, an illustrative embodiment of a syringe assembly 100 is shown. The syringe assembly 100 generally includes: a barrel 105; a needle 110; a plunger 115; a spring 120; a rear shell 125; a cap 130; a front shell 135; a second spring 140; and a needle shield 145.

The barrel 105 includes a forward end 150, a rear end 155, and a reservoir 160 for containing a medicament. In an alternative embodiment, as described in U.S. patent application Ser. No. 12/429,985, entitled “Syringe Having Extended Blending Path” by Thomas Chun, herein incorporated by reference in its entirety, the reservoir 160 may be divided into a solvent containing portion and a solute containing portion by a barrier where the barrier is selectively openable to provide fluid communication between the solute containing portion and solvent containing portion; further, a flow path member may be disposed between the reservoir 160 and needle 110 such that the flow path member establishes a flow path along which a medicament solution generated by blending the solvent and solute may flow in response to movement by the plunger 115. In another alternative embodiment, as shown in FIG. 4, the flow path member 165 may disposed within the reservoir 160 near the barrel forward end 150. In yet another alternative embodiment, the barrel 105 may be a LyoTip™ barrel arrangement 400. In yet another embodiment, as best shown in FIGS. 1-3, a flow path member 165 may be disposed within the reservoir 160 and provide fluid communication between the reservoir 160 and needle 110.

Referring again to FIGS. 1-3, the hollow needle 110 may coupled to the forward end 150 of the barrel 105 via any suitable means and is in fluid communication with the reservoir 160. In addition, the needle 110 may either be releasably or rigidly secured to the forward end 150 of the barrel 105. The needle 110 may be of any suitable length or gage for injecting a medicament into a patient. The needle 110 may be configured for subcutaneous injection, intramuscular injection, intravenous injection, or the like.

The plunger 115 has a first end 170 which may include a stopper 175 for sliding engagement within the reservoir 160 and expelling medicament through the needle 110. The plunger 115 may also include a pad 180 for receiving medicament delivery pressure from the spring 120 whereby medicament may be expelled from the reservoir 160 through the needle 110. In the illustrative embodiment, the pad 180 is disposed in at the bottom of a bore 185 in the second end 190 of the plunger 115. The pad 180 is operable to engage a first end 200 of the spring 120 whereby the spring 120 is operable to provide a medicament delivery pressure thereto. The bore 185 may also be adapted to receive a portion of the spring 120 therein. Additionally, as will be discussed below, in one embodiment, the circumference of the plunger may include an annular groove 195 for releasably receiving one or more inward projections 210 of one or more deflectable flanges 215 of the cap 130.

The spring 120 includes a first end 205 and a second end 225. As will be discussed further below, the spring 120 is operable to be compressed between the surface 225 of the cap 130 and the plunger pad 180, and, upon disengagement of the cap 130 from the plunger 115, the spring 120 is operable to provide a medicament delivery pressure to the plunger pad 180 whereby medicament is expelled through the needle 110. In one embodiment, the spring 120 is also operable to move the barrel 105 and needle 110 forward such that the needle 110 may pierce the skin of the patient prior to the spring 120 delivering the medicament delivery pressure to the plunger pad 180. In one embodiment, the spring 120 is a compression spring, although, as will be appreciated by those skilled in the art, other resilient or expandable mechanism(s) for providing a medicament delivery pressure to the plunger pad 180 may be employed.

The rear shell 125 includes an interior 235 and a step 240 therein. The rear shell 125 may be adapted to receive the cap 130 therein. Additionally, the rear shell 125 may also be adapted to receive a portion of the plunger 115 as well as the spring 120 therein. Moreover, the rear shell 125 may be adapted to be coupled, either releasably or lockingly, to the front shell 135. As will be discussed in further detail below, the step 240 may be adapted to engage one or more deflectable flanges 215 of the cap 130 to urge the cap 130 in releasable engagement with the plunger 115. In one embodiment, the step 240 engages one or more the outward projection 245 of the cap 130 to urge the cap 130 into releasable engagement with the plunger 115.

The cap 130 includes a surface 225 for engaging the second end 220 of the spring 120 such that the spring 120 may be compressed between the plunger pad 180 and the surface 225. The cap 130 may be tubular with an opening 230 adapted to receive a portion of the spring 120 and a portion of the plunger 115. The cap 130 may be adapted to releasably engage the plunger 115 such that the spring 120 may remain compressed between the surface 220 and plunger pad 180 when the plunger 115 and cap 130 are in such releasable engagement. In one embodiment, the cap 130 includes one or more deflectable flanges 215, with each flange 215 having at least one projection 210 projecting inwardly relative to the aperture 230. The inward projection(s) 210 may be adapted to be releasably received by the plunger groove 195. In one embodiment, the step 240 of the rear shell 125 biases the flange(s) 215 towards the plunger 115 such that the projection(s) 210 are releasably received by the plunger groove 195 whereby the spring 120 is maintained in a compressed state between the cap surface 225 and plunger pad 180. Additionally, as previously mentioned, each flange 215 may also include one or more outward projections 245 projecting outwardly relative to the aperture 230. Each outward projection 245 may be adapted to releasably engage the rear shell step 240 such that the step 240 biases each flange 215 in releasable engagement with the plunger 215.

The cap 130 may be moved from a first position, where the cap 130 is in releasable engagement with the plunger 115 such that the spring 120 is compressed between the cap surface 225 and the plunger pad 180, to a second position, where the cap 120 and plunger 115 are disengaged. When the cap 130 is moved to the cap second position, flange(s) 215 disengage the rear shell step 240 whereby the cap 120 and plunger 115 disengage thereby allowing the spring 120 to expand and urge the plunger 115, the barrel 105 and needle 110 forward and/or deliver medicament delivery pressure to the plunger pad 180 such that medicament is expelled through the needle 110 into the patient.

The front shell 135 includes a interior volume 250 and a step 255 therein. As will be discussed further below, the step 255 may be adapted to engage the tangs 265 of the needle shield 145 to maintain the needle shield 145 within the front shell 135 prior to an injection being delivered, as well as engage the shoulder 270 of the needle shield 145 to prohibit the needle shield 145 from completely exiting the front shell 135 after an injection has been delivered. The front shell 135 also includes an aperture 260 for allowing a portion of the needle shield 145 and needle 110 to pass therethrough. The front shell 135 may be configured to be coupled, either lockingly or releasably, to the rear shell 125. The front shell 135 may also include a shoulder 262 for engaging a barrel flange 285 to stabilize the barrel 105 and needle 110 while the spring 120 delivers medicament delivery pressure to the plunger pad 180.

The second spring 140 includes a first end 275 and a second end 280. The second spring 140 extends between the first surface 272 of the shoulder 270 of the needle shield 145 and the barrel 105. In the illustrative embodiment, the second spring 140 extends from the needle shield shoulder 270 to a barrel flange 285, but it will be appreciated that the second spring 140 may abut or engage the barrel 105 in any suitable way that permits the second spring 140 to be compressed between the barrel 105 and needle shield shoulder 270. In an alternative embodiment, the second spring 140 may abut a barrel shield (not shown) that at least partially encases the barrel.

In one embodiment, the second spring 140 is compressed between the barrel 105 and needle shield shoulder 270 as the first spring 120 moves the barrel 105 and needle 110 forward prior to the first spring 120 applying a medicament delivery pressure to the plunger pad 180. As will be discussed further below, after the delivery of an injection and upon drawing the assembly 100 away from the patient's skin, the second spring 140 is operable expand and move the needle shield 145 from a retracted position, where the needle shield 145 is substantially encased within the front shell 135, to a second or needle protecting position, where the shield portion 290 of the needle shield 145 extends through the aperture 260 to encase at least a portion of the needle 110 also extending through the aperture 260. Further, in one embodiment, the second spring 140 is a compression spring, although, as will be appreciated by those skilled in the art, other resilient or expandable mechanism(s) for moving the needle shield 145 to a needle protecting or second position after the delivery of an injection may be employed.

The needle shield 145 includes a shoulder 270, having a first surface 272 and a second surface 274, a shield portion 290 and one or more tangs 265. The needle shield 145 may be tubular with a central aperture 295. The aperture 295 may be adapted to receive a portion of the barrel 105 as well as permit a portion of the needle 110 to pass therethrough. The first surface 272 of the shoulder 270 engages the spring second end 280 and receives pressure from the second spring 140, after the second spring 140 is compressed, to move the needle shield 145 from a retracted position to a needle protecting or second position. The tangs 265 are operable to engage the front shell step 255 and maintain the needle shield 145 within the front shell 135. As the second spring 140 expands to move the needle shield 145 from the retracted position to the needle protecting position, the tangs 265 are compressed by the front shell 135 thereby allowing them to through the front shell aperture 260. Once the needle shield 145 is moved to the needle protecting position, the tangs 265 flare out and prohibit the needle shield 145 from returning to the retracted position. In one embodiment, the step portion 255 of the front shell 135 is lockingly secured between the tangs 265 and second surface 274 of the shoulder 270 when the needle shield 145 is in the needle protecting position. With respect to the shield portion 290, in the retracted position, the shield portion 290 is encased by the front shell 135; and, in the needle protecting or second position, the shield portion 290 extends through the front shell aperture 295 and at least partially encases the needle 110 also extending through the front shell aperture 295.

With particular reference to FIGS. 1-2, operation of syringe assembly 100 will be summarized. First, a front cap 300 is removed from the front shell 135 to expose the front shell aperture 260. The assembly 100 may then be brought to a patient's skin such that the front shell 135 abuts the patient's skin in such a manner that the needle 110 may pass through the front shell aperture 260 and pierce the patient's skin when the spring 120 acts upon the plunger pad 180. The cap 130 may then be depressed by a user such that the cap 130 moves from a first position, where the cap 130 releasably engages the plunger 115, to a second position, where the cap 130 disengages the plunger 115. In one embodiment, movement of the cap 130 from the first cap position to the second cap position causes one or more cap flanges 215 to disengage the rear shell step 240 whereby the cap 130 disengages the plunger 115. In one embodiment, movement of the cap from the first cap position to the second cap position causes one or more inward flange projections 210 of the cap 130 to disengage the plunger groove 195 whereby the cap 130 disengages the plunger 115.

Once the cap 130 disengages the plunger 115, the spring 120 expands. As the spring 120 expands, the barrel 105 and needle 110 are urged forward such that the needle 110 extends through the front shell aperture 260 to pierce the patient's skin. Additionally, such forward movement of the barrel 105 compresses the second spring 140 between the barrel 105 and needle shield shoulder 270. As the spring 120 continues to expand, the spring 120 delivers a medicament delivery pressure to the plunger pad 180 whereby medicament is expelled from the reservoir 160, through the needle 110, and into the patient.

After the medicament has been delivered, the assembly 100 may be withdrawn from the patient's skin. As the assembly 100 is withdrawn from the patient, the second spring 140 expands and moves the needle shield 145 from a retracted position, where the needle shield 145 is substantially encased by the front shell 135, to a needle protecting or second position, where the shield portion 290 of the needle shield 145 extends through the front shell aperture 260 and encases the needle 110 also extending through the front shell aperture 260. As the needle shield 145 moves from the retracted position to the needle protecting position, the needle shield tangs 265 are depressed by the front shell 135 and moved from within the front shell 135 through the front shell aperture 260. Once the needle shield 145 has been moved to the needle protecting positioned, the tangs 265 flare out and prohibit the needle shield 145 from returning to the retracted position.

While the present disclosure has been described in connection with what is considered the most practical and preferred embodiment, it is understood that this disclosure is not limited to the disclosed embodiments, but is intended to cover various arrangements included within the spirit and scope of the broadest interpretation so as to encompass all such modifications and equivalent arrangements.

What is claimed is:

1. A syringe assembly comprising:
a barrel including a forward end and a rear end and defining a reservoir for containing a medicament;
a hollow needle coupled to the barrel forward end and in fluid communication with the reservoir;
a plunger including a first end positioned within the reservoir and a pad for receiving medicament delivery pressure for causing the plunger to move within the reservoir to expel medicament from the reservoir;
a spring including a first end and a second end, wherein the spring first end abuts the plunger pad;
a cap including an interior surface for engaging the spring second end, the cap movable from a first position, wherein the cap is in releasable engagement with the plunger and wherein the spring is compressed between the cap interior surface and the plunger pad, and a second position, wherein the cap disengages the plunger such that the spring is capable of expanding and thereby provide a medicament delivery pressure to the plunger pad;
a needle shield positioned near the barrel forward end, the needle shield movable from a retracted position to a needle protecting position; and
a second spring including a first end engaging the barrel and a second end engaging the needle shield, the second spring capable of biasing the needle shield to the needle protecting position.

2. The syringe assembly of claim 1 further comprising a barrier disposed within the barrel to separate the reservoir into a solvent containing portion and a solute containing portion, wherein the barrier is selectively openable to provide fluid communication between the solvent containing portion and the solute containing portion.

3. The syringe assembly of claim 2 further comprising a flow path member disposed between the reservoir and needle, wherein the flow path member establishes a flow path along which a medicament solution generated by blending the solvent and the solute may flow responsively to movement of the plunger under the influence of a medicament delivery pressure.

4. The syringe assembly of claim 1 further comprising a flow path member disposed within the barrel reservoir and operable to provide fluid communication between the barrel reservoir and the needle.

5. The syringe assembly of claim 4 wherein the flow path member comprises a helical flow path for providing fluid communication between the barrel reservoir and needle.

6. The syringe assembly of claim 1 wherein the plunger includes an annular groove and wherein the cap includes at least one deflectable flange having at least one projection for releasably engaging the plunger groove.

7. The syringe assembly of claim 1 further comprising a shell including an interior and a step portion therein, wherein the cap is disposed within the shell interior and wherein the step portion releasably engages the cap in the cap first position thereby releasably securing the cap in releasable engagement with the plunger.

8. The syringe assembly of claim 7 wherein the cap includes at least one deflectable flange and a projection extending from the flange for releasably engaging the shell step.

9. The syringe assembly of claim 1 wherein the second spring biases the needle shield in the needle protecting position after the cap has been moved to the cap second position.

10. The syringe assembly of claim 1 further comprising a casing for encompassing the barrel, the needle, the plunger, the needle shield, the spring, the cap and the second spring, the casing including an open end wherein the needle shield extends at least partially through the open end when the needle shield is in the needle protecting position.

11. The syringe assembly of claim 1 wherein the needle shield locks into the needle protecting position once the needle shield has been move to the needle protecting position.

12. The syringe assembly of claim 1 wherein the spring is a compression spring.

13. The syringe assembly of claim 1 wherein the second spring is a compression spring.

14. The syringe assembly of claim 1 wherein the spring moves the barrel and needle forward prior to the providing the medicament delivery pressure to the plunger pad.

15. An apparatus for applying a medicament delivery pressure to a plunger pad of a plunger of a syringe, the apparatus comprising:
a spring including a first end and a second end, wherein the spring first end is operable to abut the plunger pad;
a shell including an interior and a first step therein; and
a cap disposed within the shell interior, the cap including a surface for engaging the spring second end, the cap movable from a first position, wherein the shell first step retains the cap in releasable engagement with the plunger such that the spring is compressed between the cap surface and plunger pad, and a second position, wherein the cap disengages the shell first step such that the cap disengages the plunger such that the spring expands to move the barrel and needle forward as well as deliver medicament delivery pressure to the plunger pad.

16. The apparatus of claim 12 wherein the spring is a compression spring.

17. The apparatus of claim 12 wherein the plunger includes an annular groove and wherein the cap includes at least one deflectable flange having at least one projection for releasably engaging the plunger groove.

18. The apparatus of claim 12 wherein the cap includes at least one deflectable flange and a projection extending from the flange for releasably engaging the shell step.

19. The apparatus of claim 12 wherein the spring is a compression spring.

20. A syringe assembly comprising:
a barrel including a forward end and a rear end and defining a reservoir within which a medicament may be contained;
a hollow needle couped to the barrel forward end and in fluid communication with the reservoir;
a plunger including a first end positioned within the reservoir and a pad for receiving medicament delivery pressure for causing the plunger to move within the reservoir to expel medicament from the reservoir;
a spring including a first end and a second end, wherein the spring first end abuts the plunger pad;
a rear shell portion including an interior and a first step therein;
a cap disposed within the shell interior, the cap including a surface for engaging the spring second end, the cap movable from a first position, wherein the shell first step retains the cap in releasable engagement with the plunger such that the spring is compressed between the cap surface and plunger pad, and a second position, wherein the cap disengages the shell first step such that the cap disengages the plunger such that the spring expands to move the barrel and needle forward as well as deliver medicament delivery pressure to the plunger pad;
a front shell portion, the front shell portion including an interior volume, a second step, and a aperture, wherein at least a portion of the needle extends through the front shell aperture when the barrel and needle are moved forward by the spring;
a second spring including a first end and a second end, wherein the second spring first end abuts the barrel; and
a needle shield disposed within the aperture of the front shell, the needle shield including an aperture for allowing the needle to pass therethrough, and a shoulder for engaging the second spring second end, the needle shield movable from a retracted position, wherein the needle shield is encased by the front shell portion, and a second position, wherein at least a portion of the needle shield extends through the front shell aperture and encompasses at least a portion of the needle extending through the front shell aperture, wherein the second spring biases the needle shield in the second position after the barrel and needle are moved forward by the spring.
